(19) United States
(12) Patent Application Publication
(10) Pub. No.: US 2009/0204155 A1
(43) Pub. Date: Aug. 13, 2009

(54) POLYAXIAL BONE ANCHOR WITH HEADLESS PEDICLE SCREW

(76) Inventor: Felix Aschmann, Basel (CH)

Correspondence Address: STROOCK & STROOCK & LAVAN, LLP, 180 MAIDEN LANE, NEW YORK, NY, 10038, US

(21) Appl. No.: 12/158,031

(22) PCT Filed: Dec. 15, 2006
(86) PCT No.: PCT/US2006/047986

§ 371 Date: Oct. 15, 2008

Related U.S. Application Data
(60) Provisional application No. 60/752,016, filed on Dec. 19, 2005.

(51) Int. Cl. A61B 17/70; A61B 17/86

(52) U.S. Cl. 606/264; 606/301; 606/305

(57) ABSTRACT

A polyaxial bone anchor has a headless anchor member (e.g., a screw, hook, or other structure for attaching to bone) that allows the size of the bone anchor to be small. A locking element securely snap-fits over the headless anchor member such that inadvertent separation from the anchor member is unlikely. When the anchor member is attached to the locking element and the locking element is seated within the anchor head of the bone anchor, the headless anchoring member can polyaxially rotate about a central axis of the bone anchor before being locked in place.

### TECHNICAL FIELD OF THE INVENTION

This invention relates to bone fixation devices and related methods of fixation. More particularly, this invention relates to polyaxial bone anchors having headless screws and hooks, and more specifically, polyaxial pedicle screws and hooks, for use in, for example, the fixation of the spine.

### BACKGROUND OF THE INVENTION

Polyaxial bone anchors and methods of use in treating spinal disorders are known. Typical methods involve anchoring at least two screws or hooks into the vertebrae, and fixing the screws or hooks along a spinal rod to position or immobilize the vertebrae with respect to one another. The screws or hooks (referred to hereinafter as anchor members) commonly have anchor heads with U-shaped channels in which the spinal rod is inserted and subsequently clamped by a fastener, such as, for example, a threaded nut, set screw, or locking cap. These methods commonly involve multiple anchor members and multiple spinal rods. The spinal rods may be shaped to maintain the vertebrae in a desired orientation so as to correct the spinal disorder at hand (e.g., to straighten a spine having abnormal curvature). Additionally or alternatively, anchor members may be spaced along the rods(s) to compress or distract adjacent vertebrae or bilaterally move vertebrae.

Surgeons may encounter difficulty with spinal fixation and stabilization methods because of difficulty aligning the spinal rod(s) with the U-shaped channels in the anchor heads. For example, anchor heads are often out of alignment with one another because of the curvature of the spine or the size and shape of each vertebrae. To facilitate easier insertion of the spinal rods into the U-shaped channels, and to provide additional flexibility in the positioning of the spinal rods and the anchor members, bone anchors have been developed where the anchor member and anchor head can initially pivot or rotate with respect to each other. These bone anchors are sometimes referred to as polyaxial bone anchors and the pivot or rotation of the anchor member is referred to as angulation.

A disadvantage of many polyaxial bone anchors is their large size, particularly that of the anchor head, which tends to be large in order to accommodate the typically bulbous or ball-shaped end of the anchor member. These anchor member ends, often referred to as the “head” of the anchor member, provide known anchor members with their polyaxial capability. However, such large polyaxial bone anchors may have limited application in view of the confined space around the human spine. It may therefore be advantageous to provide smaller polyaxial bone anchors.

### SUMMARY OF THE INVENTION

The invention is directed to polyaxial bone anchors and methods of use for attaching a rod, such as a support or spinal rod, to a bone, such as a vertebra. The bone anchor may include a hollow, generally cylindrical housing, body, or head (referred to hereinafter as an anchor head), a headless anchor member (such as, for example, a pedicle screw, hook, or other structure for attaching to bone), an internal locking element, an optional hollow generally cylindrical internal sleeve, and an optional fastener. The fastener may be a threaded outer ring with set screw, but alternatively, may be of other types or arrangements, such as, for example, a locking cap with set screw, a threaded nut, or a locking sleeve mounted on or over the top portion of the anchor head. The anchor head and optional internal sleeve may have a U-shaped channel for receiving a support/spinal rod (referred to hereinafter as a spinal rod or rod). The locking element preferably is sized and shaped to snap-fit onto the headless anchor member. The fastener may close the top opening of the U-shaped channel after a rod has been placed therein and, in combination with the locking element, lock or clamp the respective positions of the anchor member and spinal rod with respect to each other and the anchor head.

The headless anchor member has a shank with a non-threaded portion. In the case of a pedicle screw, the shank also has a threaded portion for insertion into bone. In the case of a pedicle hook, the shank is integrally or discretely connected to a hook structure that attaches to bone. Similarly, for other types of anchor members, the shank with the non-threaded portion may be integrally or discretely connected to other structures for attaching to bone.

The non-threaded shank portion preferably has a circular cross section, but may be of other cross-sectional shapes, such as, for example, polygonal. The non-threaded portion preferably has a constant diameter or width throughout its length and preferably has an external groove around its circumference or perimeter (the groove is not part of any thread). The non-threaded portion is not integrally connected to an enlarged end (e.g., a spherically or partially spherically shaped end). Such an enlarged end is often referred to as the “head” of the anchor member. Anchor members of the invention may thus be referred to as “headless.”

The locking element, internal sleeve, and anchor head have features that allow the locking element to rotate and/or pivot within the anchor head. This in turn allows the anchor member to angulate in all directions around and away from a central axis running through a bottom opening in the anchor head. The anchor member may then be locked at a desired angle and direction with respect to the anchor head.

The locking element, which may be a collet or collet-styled bushing, has a spherical or at least a partially spherical exterior shape. This spherical exterior shape allows the locking element to rotate and/or pivot within the anchor head, which in turn allows the anchor member to angulate in all directions about the central axis. The interior area of the locking element is sized and shaped slightly smaller than the size and shape of the non-threaded shank portion of the anchor member in order that the locking element be snap-fitted over the non-threaded shank portion. The locking element has a plurality of resilient fingers that radially expand to initially receive the non-threaded shank portion. Preferably, each finger has an interior ridge that sits in the external groove of the non-threaded shank portion when that portion is fully received in the locking element. This ridge-groove feature lessens the likelihood of the anchor member inadvertently separating from the locking element. The locking element preferably is configured to receive at least the uppermost portion of the non-threaded shank portion, with the remaining portion of the anchor member extending through the bottom opening of the anchor head.

The generally cylindrical internal sleeve has a bottom surface that preferably tapers or curves inward and upward such that when positioned in the anchor head it contacts the top exterior surface of the locking element fingers. This facilitates locking of the anchor member to the locking element, while allowing pivoting or rotation of the locking element prior to locking. In particular, when the anchor member is ready to be locked (i.e., the anchor member is positioned as desired), the fastener is tightened, causing the bottom surface of the internal sleeve to press down on the outside of the locking element fingers, which compress around the non-threaded shank portion to lock the position of the anchor member.

The anchor head has a lower portion with an interior surface around the bottom opening that is preferably tapered or spherically or partially spherically shaped to substantially match and contact a portion of the exterior surface of the locking element. This also facilitates pivoting and/or rotation of the locking element within the anchor head prior to locking.

By not requiring the typically large heads of known anchor members in order to provide polyaxial capability, a polyaxial bone anchor, and in particular its anchor head, can have a small size. This small size advantageously improves the versatility of the bone anchor.

### BRIEF DESCRIPTION OF THE DRAWINGS

The detailed description will be better understood in conjunction with the accompanying drawings, in which like reference characters refer to like parts throughout, and in which:

FIGS. 1-3 are perspective, partial cutaway perspective, and exploded perspective views, respectively, of a known polyaxial bone anchor;

FIGS. 4 and 5 are perspective and partial cutaway perspective views of a polyaxial bone anchor with a headless pedicle screw according to the invention;

FIG. 6 is an exploded perspective view of the polyaxial bone anchor of FIGS. 4 and 5; and

FIG. 7 is an enlarged partial cutaway view of the anchor head, internal locking element, and headless anchor member of the polyaxial bone anchor of FIGS. 4-6.

### DETAILED DESCRIPTION OF THE INVENTION

The invention can be used to treat various spinal disorders including, for example, degenerative and other instabilities due to decompression, tumors, infections, and fractures.

Note that while the polyaxial bone anchor is described and illustrated herein with reference to certain preferred or exemplary embodiments, the invention should not be limited to those preferred or exemplary embodiments. Furthermore, the features described and illustrated herein can be used singularly or in combination with other features and embodiments.

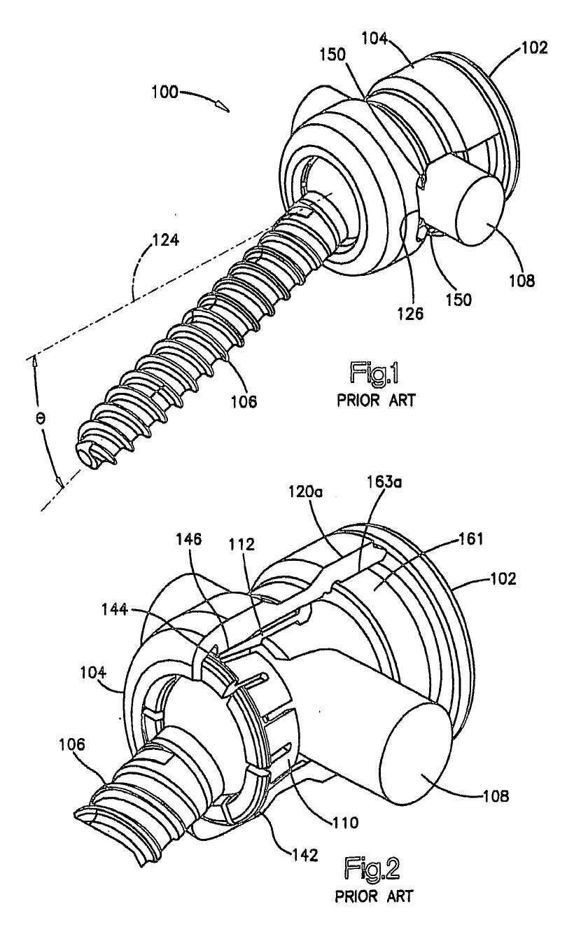

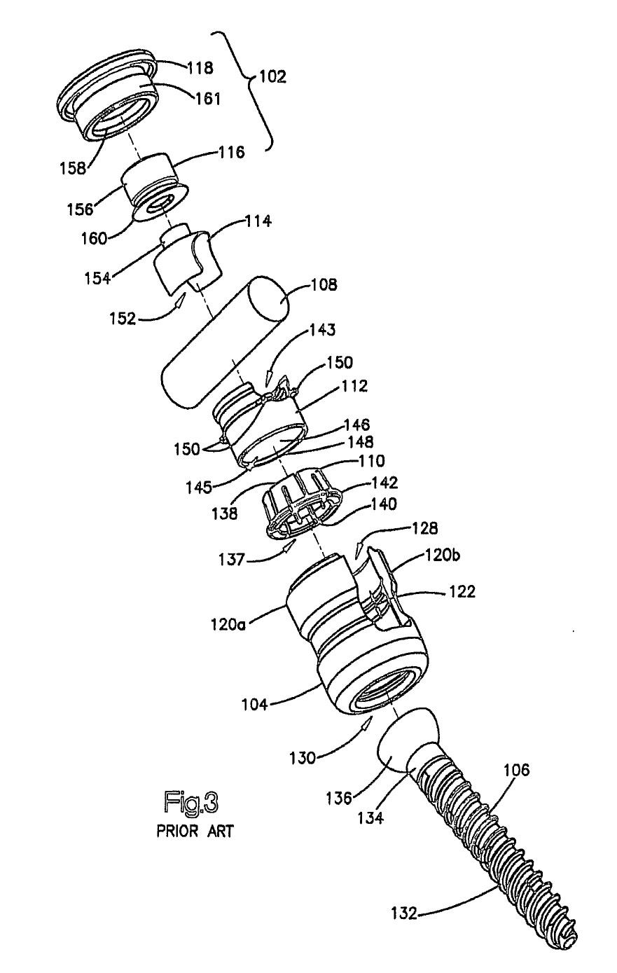

FIGS. 1-3 show a known polyaxial bone anchor. Polyaxial bone anchor 100 includes a fastener 102, an anchor head 104, and an anchor member 106. A spinal rod 108 may be clamped or locked in bone anchor 100, while anchor member 106, which may be a pedicle screw, hook, or other similar structure (and is referred to hereinafter as pedicle screw 106) may be inserted into or attached to bone. Bone anchor 100 may also include a locking element 110, an internal sleeve 112, and a saddle 114. Fastener 102 may include a set screw 116 and a threaded outer body 118.

Anchor head 104 is cylindrically hollow having a longitudinal bore 122 along longitudinal axis 124. Longitudinal bore 122 is bounded by a top opening 128 and a bottom opening 130 in anchor head 104. Anchor head 104 also has a generally U-shaped opening 126 transverse to longitudinal bore 122 for receiving spinal rod 108 or other similar part.

Pedicle screw 106 has a shank 132, neck 134, and head 136. Neck 134 may have a smaller diameter or width than shank 132, while head 136 has a gradually increasing diameter or width along the length of the screw until its maximum diameter or width is reached at or near the top of the screw. Although head 136 is frusta-spherical, many known embodiments of pedicle screws have fully or substantially spherical or bulbous heads, in which case the diameter or width along the screw length decreases from its maximum until the top of the screw is reached. Head 136 of pedicle screw 106 may also be partially spherically shaped.

Locking element 110 may be a collet or collet-styled bushing having a large resilient bottom opening 137. The diameter of the bottom opening is slightly less than the diameter of head 136. Locking element 110 also has a plurality of resilient fingers 138. The resiliency of opening 137 and fingers 138 is provided in large part by slots 140. Opening 137 and fingers 138 both expand to allow the enlarged head 136 of pedicle screw 106 to be inserted within locking element 110. As shown, slots 140 may alternately extend from each end of locking element 110. While the arrangement, shapes, and dimensions of the slots and fingers may be different in other known locking elements, they generally perform substantially they same function: to initially expand, hold, and then lock in place the pedicle screw. Locking element 110 may also have a lip 142 that snaps into a groove 144 on an interior surface of the lower portion of anchor head 104. Groove 144, which is not a part of a thread, holds locking element 110 in place within anchor head 104. The pedicle screw alone may be implanted in bone first, and the anchor head/locking element assembly snap-fitted over the screw head thereafter. With locking element lip 142 snapped into anchor head groove 144, and screw head 136 snapped into locking element 110, the neck and shank of pedicle screw 106 extend out of bottom opening 130 of anchor head 104. Prior to being locked or clamped in place, head 136 provides pedicle screw 106 with the capability of polyaxially (or angularly) rotating or pivoting by an angle θ around central axis 124. Angle θ in known polyaxial bone anchors is typically about 10-15° (i.e., the angular rotation of pedicle screw 106 forms a cone of typically about 20-30°), although angle θ may also extend to 25-30° resulting in an angular rotation that forms a cone of about 60° or less.

Optional internal sleeve 112 may be inserted downward through top opening 128 into anchor head 104. Sleeve 112 is hollow and generally cylindrical and may have a generally U-shaped channel 143 for receiving a rod. Sleeve 112 has a bottom opening 145 that fits over locking element fingers 138. If pedicle screw 106 includes sleeve 112, the outside surface of the locking element, or at least a portion thereof, may interact with the interior surface 146 of sleeve 112, instead of interacting with the interior of anchor head 104. The portion of interior sleeve surface 146 positioned over fingers 138 may be tapered to facilitate locking. The outside or exterior surface of the locking element may have a tapered surface to correspond to the interior surface of the sleeve. Bottom edge 148 of sleeve 112 may be positioned above or rest on lip 142 of locking element 110. Sleeve 112 may further have a pair of tabs 150 on each side of its U-shaped channel that sit at the bottom corners of U-shaped opening 126 of anchor head 104 to align the U-shaped channel with the U-shaped opening.

Optional set screw 116 may have external threads 156 that mate with internal threads 158 of outer body 118. Set screw 116 may also have a flared bottom 160 to prevent it from being screwed out of outer body 118. Set screw 116 may further be preloaded into outer body 118 before fastener 102 is attached to anchor head 104.

Optional saddle 114 has an upper portion 154 that fits into set screw 116. The set screw may rotate relative to and over saddle 114. Saddle 114 facilitates clamping or locking of rod 108. A recess 152 in saddle 114 has a radius of curvature that may be the same as, or slightly smaller than, spinal rod 108. As set screw 116 is driven downward, it pushes and then clamps saddle 114 over and onto spinal rod 108.

With a spinal rod in the U-shaped channel and saddle 114 either positioned in anchor head 104 on top of the spinal rod or inserted into set screw 116, fastener 102 may be attached to anchor head 104 by first threading outer body threads 161 into anchor head threads 163a,b on respective interior surfaces of upper arms 120a,b of the anchor head. This closes top opening 128. At this stage, bone anchor 100 is unlocked, meaning that pedicle screw 106 is free to angulate and rod 108 is free to slide in and out of U-shaped opening 126 (although it can no longer be removed through top opening 128). Upon satisfactory positioning of the pedicle screw and rod, set screw 116 may be driven downward to lock the screw and rod in place. As set screw 116 is driven downward, saddle 114 contacts rod 108, pushing it downward. Then, depending on the design of the bone anchor, rod 108 may then contact sleeve 112, pushing it down against locking element 110, or saddle 114 and/or rod 108 may push down on locking element 110. In any case, contact with locking element 110 causes resilient fingers 138 to compress around and ultimately crush-lock head 136 of pedicle screw 106, clamping both the pedicle screw and spinal rod in place.

Note that while the parts shown in FIGS. 1-3 are merely representative of known polyaxial bone anchors—the exact arrangement, shapes, and connection of such parts may vary—the assemblage of such known polyaxial bone anchor parts is likely to result in a large, bulky bone anchor. For example, representative exemplary dimensions of a known bone anchor 100 may include an anchor head height of about 12 mm, a width of about 10 mm, and a depth of about 8.5 mm. Pedicle screw 106 may have a shank diameter of about 4 mm, a neck diameter of about 2.75 mm, and a maximum head diameter of about 5.4 mm. Alternatively, other known bone anchors may be of other similarly large dimensions.

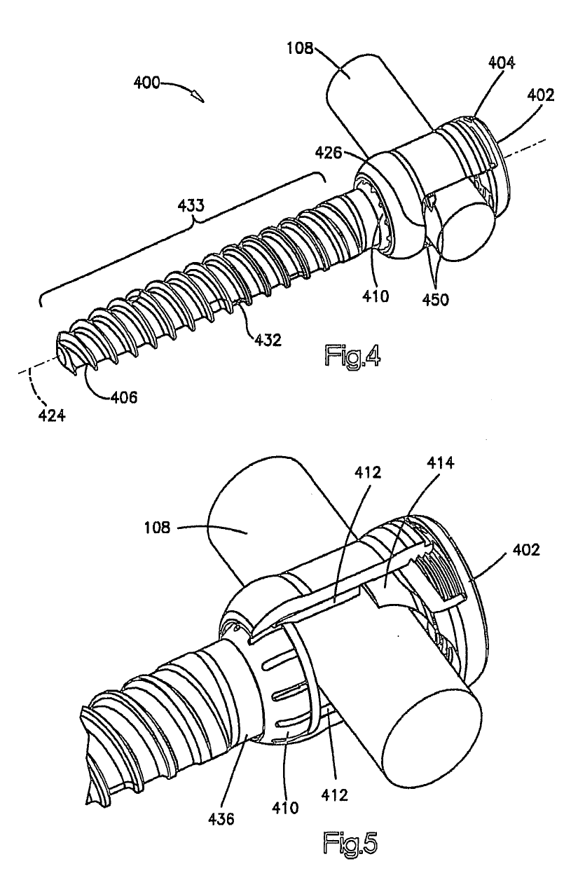

FIGS. 4-7 show a polyaxial bone anchor with a headless pedicle screw. Polyaxial bone anchor 400 preferably includes optional fastener 402, anchor head 404, headless pedicle screw 406, internal locking element 410, optional internal sleeve 412, and optional saddle 414. Anchor head 404, locking element 410, and internal sleeve 412 are designed such that headless pedicle screw 406 can polyaxially rotate with respect to anchor head 404. One or more polyaxial bone anchors 400 may be attached to, for example, the vertebrae via respective anchor members 406, and a spinal rod 108 or other similar part can be inserted into the U-shaped openings 426 of respective anchor heads 404. The spinal rod may thereafter be locked with respect to anchor heads 404. A system of bone anchors and rods can be used to correctly align the spine or treat other spinal disorders.

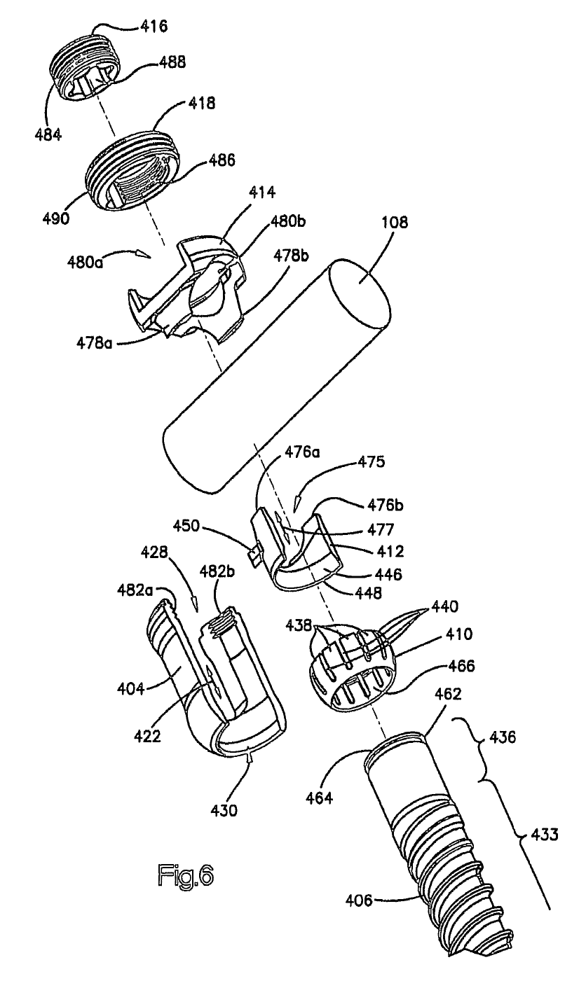

FIG. 6 shows the various parts of bone anchor 400, which includes outer ring 418 and set screw 416 of optional fastener 402, optional saddle 414, spinal rod 108, optional internal sleeve 412, locking element 410, headless pedicle screw 406, and anchor head 404. Anchor head 404 is preferably cylindrically hollow having a longitudinal bore 422 along longitudinal axis 424. Longitudinal bore 422 is bounded by a top opening 428 and a bottom opening 430 in anchor head 404. Anchor head 404 also has a generally U-shaped opening 426 transverse to longitudinal bore 422 for receiving spinal rod 108 or other similar part.

Headless pedicle screw 406 has a shank 432 with a threaded portion 433 and a non-threaded portion 436. The threaded portion is for insertion into bone. The non-threaded portion preferably has a circular cross section, but alternatively may have other cross-sectional shapes, such as, for example, polygonal. The non-threaded portion also has a preferably constant diameter throughout its length and preferably has an external groove 462 around its circumference. Non-threaded portion 436 is not integrally connected to a “head,” i.e., a top portion that expands outward (e.g., a spherically or partially spherically shaped portion) or any other structure that is otherwise enlarged (e.g., a circumferential bead or lip). The top 464 of pedicle screw 406 preferably has a recess or slot (not shown) keyed to receive a hex wrench, torque wrench, or other known driver or tool to implant the pedicle screw by rotating into, for example, a bone such as a vertebra.

Polyaxial bone anchor 400 may first be assembled by snap-fitting locking element 410 over non-threaded portion 436 of headless pedicle screw 406 and inserting that assembly screw-shank first through top opening 428 of anchor head 404. Locking element 410 is configured to receive at least the uppermost portion of non-threaded portion 436, with the remaining portion of pedicle screw 406 (i.e., threaded portion 433 and lowermost portion 436b) extending through bottom opening 430 of anchor head 404. Alternatively, headless pedicle screw 406 may be inserted into bone and thereafter locking element 410 may be assembled to non-threaded portion 436 of the pedicle screw.

Locking element 410 may be described as a collet or collet-styled bushing (referred to hereinafter as collet 410) made of a resilient material that can be compressed around the non-threaded portion of pedicle screw 406 to retain pedicle screw 406 securely in place. Preferably, the material of the collet is softer than the material of internal sleeve 412 and pedicle screw 406. Interior area 466 of collet 410 is sized and shaped to be preferably slightly smaller than the size and shape of non-threaded shank portion 436, such that the collet has to be pressed over the non-threaded portion in a friction fit. Collet 410 has a plurality of resilient fingers 438 preferably tapered inward that can radially expand or deflect outward to receive non-threaded portion 436 within interior area 466. Fingers 438 are created by slots 440, which extend from the top end of collet 410 (the top being defined as facing anchor head top opening 428). Slots 440 may have a radius or circular shaped portion (not shown) to provide stress relief and/or greater resiliency to fingers 438. The arrangement, shapes, and dimensions of the fingers and slots may be alternatively different than shown.

Preferably each, and at least two, of fingers 438 have an interior ridge 468 that snaps into external groove 462 of the non-threaded shank portion when that portion is fully received in the collet. This ridge-groove feature lessens the likelihood of headless pedicle screw 406 inadvertently separating from collet 410 during angulation or other manipulation.

Collet 410 preferably has a spherical or at least a part-spherical exterior shape. This exterior shape advantageously allows collet 410 to rotate or swivel about central axis 424 within anchor head 404 prior to the locking of the pedicle screw. The exterior surface of collet 410 contacts and is movable and preferably pivotable with respect to lower-portion inner surface 470 of anchor head 404. This in turn allows pedicle screw 406 to angulate in all directions with respect to anchor head 404. The geometry of the collet, being generally spherically or partially spherically shaped, provides in large part the polyaxial capability, thus allowing the pedicle screw to go “headless.” Note that collet 410 has no projections, grooves, lips, or other features that fix collet 410 to the anchor head.

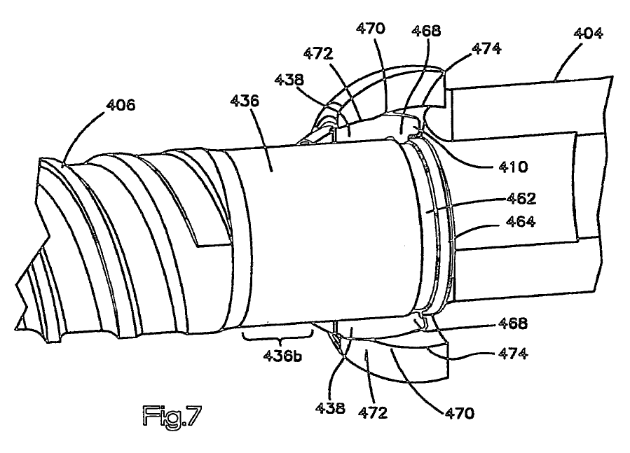

Further facilitating the angulation capability of pedicle screw 406 is the preferably tapered inner surface 470 of anchor head 404. Inner surface 470 preferably has two radii of curvature, as shown in FIG. 7. The first radius of curvature at inner surface 472 substantially matches that of the spherical exterior surface of collet 410 and allows collet 410 to contact, rotate, and pivot in anchor head 404. The second radius of curvature or conical taper at inner surface 474 is preferably greater than that of the collet's spherical exterior surface to allow the bottom edge 448 of internal sleeve 412 to fit between the collet and inner surface.

Internal sleeve 412 may be inserted downward into anchor head 404 through top opening 428, and may be preassembled in anchor head 404. Internal sleeve 412 is hollow and generally cylindrical having a longitudinal bore 477 there through. Internal sleeve 412 preferably has a generally U-shaped channel 475 for receiving a rod. U-shaped channel 475 is transverse to the longitudinal bore. Internal sleeve 412 may also have one or more tabs 450 on each outer side of U-shaped channel 475. The tabs align the sleeve in anchor head 404 and are positioned respectively on each side of U-shaped opening 126. When sleeve 412 is positioned in the anchor head, the lower portion of the sleeve fits between anchor head inner surface 474 and the exterior surface of collet fingers 438. Sleeve 412 has an inner bottom surface 446 that preferably tapers or curves inward and upward to preferably match the contour of the exterior surface of collet fingers 438. This facilitates pivoting or rotation of collet 410 prior to locking. To lock the angulation of the bone anchor (e.g., when the pedicle screw and spinal rod are positioned as desired), the sleeve is moved toward bottom opening 430 of the anchor head. As the sleeve moves downward, the sleeve's tapered inner bottom surface 446 presses on the exterior surface of fingers 438 to compress them around the non-threaded shank portion, locking the position of the pedicle screw. This compression not only acts to lock pedicle screw 406 in anchor head 404, but also serves to keep collet 410 attached to pedicle screw 406.

With fastener 402 removed from the assembly of anchor head 404, internal sleeve 412, collet 410, and headless pedicle screw 406, the pedicle screw may be attached to a bone. A tool, such as a hex wrench, torque wrench, or other known driver, may be inserted through the aforementioned assembly into the recess or slot at the top of pedicle screw 406. The screw may then be rotated, implanting it in, for example, a bone such as a vertebra.

Anchor head 404 may now be aligned to receive a rod 108. In one embodiment of the invention, rod 108 is preferably snapped into internal sleeve 412. The distance between upright arms 476a,b of sleeve 412 across U-shaped channel 475 is preferably slightly less than the diameter of rod 108. In this manner, the sleeve may provisionally retain the spinal rod but still permit the rod to slide in or be removed from the U-shaped channel. Alternatively, the distance between upright arms 476a,b may be slightly greater than the diameter of rod 108 and the rod may simply be placed in U-shaped channel 475.

With a spinal rod in the U-shaped channel, optional saddle 414 may be placed in anchor head 404 such that oppositely-positioned saddle legs 478a,b straddle rod 108 and oppositely-positioned openings 480a,b on the upper portion of saddle 414 face respective internal threads 482a,b on anchor head 404.

Set screw 416 has external threads 484 that mate with internal threads 486 of outer ring 418. Preferably, set screw 416 is preloaded into outer ring 418 before fastener 402 is attached to anchor head 104, and preferably set screw 416 cannot be screwed out of outer ring 418 (set screw 416 may have, for example, a flared bottom (not shown) to prevent it from being screwed out). Set screw 416 preferably has a star socket 488 or other type of socket or recess keyed to a known driver or tool. Preferably, the same tool or driving mechanism used to attach outer ring 418 to anchor head 404 can be used in a continuous action to further rotate set screw 416.

Fastener 402 (i.e., set screw 416 preferably preloaded into outer ring 418) may now be placed on anchor head 404, closing the U-shaped channel. Outer ring 418 attaches to anchor head 404 by engaging internal threads 482a,b on anchor head 404 with its external threads 490 through respective openings 480a,b in saddle 414. As outer ring 418 is screwed down into anchor head 404, it pushes down on saddle 414, which in turn causes saddle 414 to push down on rod 108 and the bottoms of saddle legs 478a,b to push down on respective upright arms 476a,b of sleeve 412. Inner bottom surface 446 and bottom edge 448 of sleeve 412 then press down on collet fingers 438 until, in one embodiment, sleeve 412 can no longer move downward in the space between anchor head inner surface 474 and collet fingers 438, or, in another embodiment, tabs 450 contact the bottom edge of U-shaped opening 426 on anchor head 404. Pedicle screw 406 is now provisionally locked in place, while rod 108 can still slide in and out of U-shaped channel 475. Note that placement of rod 108 in the U-shaped channel is not required to provisionally lock pedicle screw 406 in place. That is, rod 108 may be inserted in U-shaped channel 475 after pedicle screw 406 has been provisionally locked in place.

At this point, outer ring 418 can no longer be rotated downward and rod 108 can still be positioned (e.g., moved) relative to anchor head 404 and pedicle screw 406. Upon satisfactory positioning of rod 108, set screw 416 can be driven downward to lock the rod in place in the anchor head. As set screw 416 is driven downward, it contacts and presses down on rod 108. Rod 108 in turn moves down the bore of the anchor head until it contacts and presses down on pedicle screw top 464. Further downward rotation of set screw 122 applies pressure to the spinal rod, clamping the rod in a final position in anchor head 404 such that the rod cannot slide and/or be removed from the anchor head. The downward pressure applied by the rod on pedicle screw top 464 may further compression lock pedicle screw 406 by tightly wedging collet 410 between anchor head inner surface 472 and the non-threaded shank portion.

Alternatively, pedicle screw 406 can be locked with respect to the anchor head by placing rod 108 in the anchor head and pushing down on rod 108 so that rod 108 pushes down on sleeve 412. This in turn compresses collet 410 and locks (i.e., prevents) the angulation of the pedicle screw in the anchor head. With this method, the fastener and saddle do not have to be engaged or connected to the anchor head to lock the position of the pedicle screw relative to the anchor head—yet, a user can move or remove rod 108. Alternatively, the fastener and saddle can be applied to the anchor head while the force to lock the angulation of the pedicle screw is applied by a user to rod 108.

Although fastener 402 is shown as having external threads 480, fastener 402 may instead be a non-threaded locking cap similar or identical to that described in U.S. Provisional Patent Application No. 60/674,877, filed Apr. 25, 2005, which is incorporated herein by reference in its entirety. Saddle 414 may also be attached to fastener 402 as part of an assembly. Alternatively, fastener 402 may be of other types, and anchor head 404 may have corresponding features required to permit attachment and operation of fastener 402.

Note that collet 410 may be advantageously used with other headless anchor members and that the assembly of collet 410 and headless pedicle screw 406 may be advantageously used with other types of anchor heads, internal sleeves, and fasteners than those shown herein. For example, collet 410 and screw 406 may be used with the bone anchor disclosed in the previously cited U.S. Provisional Patent Application No. 60/674,877, filed Apr. 25, 2005, incorporated herein by reference in its entirety.

1-17. (canceled)

18. A polyaxial bone anchor for attaching a rod to a bone comprising:
an anchor head comprising a longitudinal bore having a top opening and a bottom opening, a generally U-shaped channel transverse to the longitudinal bore for receiving the rod, and a central axis extending through the bore;
a headless anchor member having a top, a bottom, and a shank, the shank having a non-threaded portion adjacent to the top of the anchor member, the non-threaded portion having a substantially constant diameter, and a bone engaging portion adjacent to the bottom of the anchor member, the bone engaging portion of the anchor member extending through the bottom opening of the anchor head for attaching to bone;
a locking member configured to be retained within the anchor head and to receive at least a portion of the non-threaded portion of the shank, the non-threaded portion of the shank includes an exterior groove adjacent to the top of the anchor member and the locking member has an interior ridge positionable in the groove of the non-threaded portion of the shank when the non-threaded portion of the shank is fully received within the locking member, the locking member operative to angulate about the central axis in all directions; and
a fastener removably mountable to the anchor head to close the top opening of the bore and to lock both the position of the rod in the U-shaped channel and the angulation of the locking member.

19. The polyaxial bone anchor of claim 18, wherein the bone engaging portion of the shank is threaded.

20. The polyaxial bone anchor of claim 18, wherein the bone engaging portion of the shank is a hook.

21. The polyaxial bone anchor of claim 20, wherein the hook is integrally formed with the non-threaded portion of the shank.

22. The polyaxial bone anchor of claim 18, wherein the locking member contacts the anchor head and rotates within the anchor head prior to the fastener locking the positions of the rod and anchor member.

23. The polyaxial bone anchor of claim 18, wherein the locking member comprises a collet having a plurality of resilient fingers for compressing against the non-threaded portion of the shank, at least two of the fingers having the interior ridge.

24. The polyaxial bone anchor of claim 18, wherein the locking member has at least a part spherical exterior shape.

25. The polyaxial bone anchor of claim 18, wherein the locking member has an interior area sized and shaped to substantially match the size and shape of the non-threaded portion of the shank.

26. The polyaxial bone anchor of claim 18, further comprising a hollow sleeve having a generally U-shaped channel for receiving the rod, the sleeve retained within the anchor head.

27. The polyaxial bone anchor of claim 26, wherein the sleeve has a bottom surface that engages the locking member.

28. The polyaxial bone anchor of claim 18, wherein when the spinal rod is locked in position in the U-shaped channel, the spinal rod contacts the top of the locking member.

29. The polyaxial bone anchor of claim 18, wherein the anchor head has an interior surface around the bottom opening that contacts a portion of the exterior surface of the locking member.

30. The polyaxial bone anchor of claim 18, wherein the anchor head has a tapered interior surface around the bottom opening that contacts and substantially matches the contour of a portion of the exterior surface of the locking member.

31. The polyaxial bone anchor of claim 18, wherein the fastener is a locking cap comprising a locking ring and set screw.
